(19) United States
(12) Patent Application Publication
(10) Pub. No.: US 2022/0008253 A1
(43) Pub. Date: Jan. 13, 2022

(54) WOUND DRESSING HAVING MICROBE RESISTANT MICROPATTERENED SURFACE

(71) Applicant: Hannah Herbst, Boca Raton, FL (US)

(72) Inventor: Hannah Herbst, Boca Raton, FL (US)

(21) Appl. No.: 16/922,857

(22) Filed: Jul. 7, 2020

(51) Int. Cl. A61F 13/00

(52) U.S. Cl. CPC A61F 13/00021; A61F 2013/00119; A61F 13/00987

(57) ABSTRACT

A wound dressing includes a dressing member or body which has a wound-facing side intended to be placed adjacent a wound, such a surgical wound. The wound-facing side of the wound dressing is provided with a plurality of microstructures in the form of denticles. Each of the denticles includes a body that extends from a base of the denticle laterally over, but not connected to, the dressing member. The denticles are arranged in an overlapping manner such that the body of one denticle overlaps the body of one or two other adjacent denticles. The denticles create a microstructured surface that resists bioadhesion, and which, upon flex of the wound dressing, create mechanical interference with each other that can dislodge organisms that manage to adhere to the wound-facing side of the wound dressing in spite of the microstructured surface.

### FIELD OF THE INVENTION

The present invention relates generally to micropatterning of surfaces, and, more particularly, relates to wound care dressings having microbe resistant micropatterned surfaces.

### BACKGROUND OF THE INVENTION

A standard regimen for facilitating healing of wounds is to apply a dressing. The dressing typically consists of a woven or unwoven fibrous material that is placed over the wound and secured in place. The function of the dressing is, in part, to support the surrounding tissue to reduce mechanical stress on the would site in order to facilitate the fastest healing. Although dressings are sterile when applied, as a result of exposure to bodily fluids, they can become fouled and a source of pathogens. Accordingly, wound dressings need to be changed periodically to reduce the chance of infection. This is especially true in hospitals where so called “superbug” pathogens are found. These pathogens are resistant to antibiotics and are virtually incurable. Surgical site infection (SSI) and healthcare associated infection (HAI) are among leading causes of mortality, and are correspondingly associated with increased medical expense.

One factor in the prevalence of SSI/HAI is the formation of biofilm on a given surface. The surface is one that is moist or wet, and allow microbes to adhere (bio-adherence) to the surface. Wound sites tend the be moist, and conventional wound dressings can provide a high level of bio-adherence. Microbe bio-adherence is also an issue in marine applications. In particular, on the submerged exterior surface of boat and ship hulls. A great deal of effort has gone into reducing “fouling” of these surfaces to maintain optimum efficiency while moving through the water. Various approaches are used to reduce marine fouling, such as the use of surface materials that chemically repel microbes. Another approach that has been used is the micropatterning of hull surfaces. Micropatterning refers to creating micro-structural features on the surface that prevent bio-adhesion by interrupting the van der Waals force, steric interactions, and electrostatic interactions required for microbes to sufficiently adhere to a surface and create a biofilm. The microstructural features commonly use extend outward perpendicularly to the general plane of the surface by a height on the order of tens of microns to sub-millimeter dimensions. In some applications it is known to vary the height among adjacent features in patterns (e.g. “micropattern”). However, while micropatterning in marine applications is known, it is not known to apply this feature to wound dressings.

Therefore, a need exists to overcome the problems with the prior art as discussed above.

### SUMMARY OF THE INVENTION

In accordance with embodiments of the invention disclosure, there is provided a wound dressing that includes a dressing member made of a supple non-textile material and having a wound-facing side and an outward facing side opposite the wound-facing side. The wound dressing further includes a plurality of denticles formed on the wound-facing side. Each denticle of the plurality of denticles is formed to have a base portion that joins the denticle to the wound-facing side of the dressing member. The base portion of each denticle has a front and a rear. Each denticle includes a denticle body that extends upward relative to the base portion, and which extends laterally to the rear and beyond the rear of the base portion.

In accordance with another feature, for each denticle of the plurality of denticles, no portion of the denticle body extends forward of the front of the base portion of the denticle.

In accordance with another feature, for each denticle of the plurality of denticles, the denticle body comprises at least one ridge formed in the denticle body which extends from the front of the base portion to the rear along an upper surface of the denticle body.

In accordance with another feature, the at least one ridge is a plurality of ridges, wherein the plurality of ridges are formed by a plurality of concave valleys formed in the denticle body which extend from the front of the base portion to the rear along the upper surface of the denticle body.

In accordance with another feature, for each denticle of the plurality of denticles, the denticle body extends rearward to a tip that is located to the rear from the rear of the base portion by a distance that is at least that of a width of the base portion from the front to the rear of the base portion.

In accordance with another feature, the plurality of denticles are commonly oriented and arranged in rows of denticles.

In accordance with another feature, the rows are staggered such that the denticle bodies of denticles in a first row extend to rear between denticles in a second row to the rear of the first row.

In accordance with embodiments of the invention disclosure, there is provided a method of forming a wound dressing that includes forming a negative mold, having a top surface, with a plurality of denticle cavities formed in the top surface and extending in a body of the negative mold. Each denticle cavity of the plurality of denticle cavities is configured to form a denticle having a base portion at an opening in the top surface of the negative mold, and having a front and a rear. Further each denticle cavity is configured to form a denticle body that extends from the base portion to the rear beyond the rear of the base portion and is separated from the top surface of the negative mold. The method further includes pouring a dressing material onto the mold that fills in the plurality of denticle cavities to a selected height above the top surface of the negative mold, and curing the dressing material to produce a cured wound dressing. Additionally, the method includes removing the cured wound dressing from the negative mold. The cured would dressing includes a plurality of denticles corresponding to the plurality of denticle cavities.

In accordance with another feature, pouring the dressing material comprises pouring an elastomer material.

In accordance with another feature, pouring the elastomer material comprises pouring polydimethylsiloxane elastomer.

In accordance with another feature, forming the negative mold comprises forming the plurality of denticle cavities in staggered rows in the top surface of the negative mold.

In accordance with another feature, forming the negative mold comprises forming the plurality of denticle cavities such that, for each denticle cavity the corresponding denticle formed by the denticle cavity comprises at least one ridge formed in the denticle body which extends from the front of the base portion to the rear along an upper surface of the denticle body.

In accordance with another feature, forming the negative mold comprises forming the plurality of denticle cavities such that, for each denticle cavity the corresponding denticle formed by the denticle cavity comprises a plurality of ridges, wherein the plurality of ridges are formed by a plurality of concave valleys formed in the denticle body which extend from the front of the base portion to the rear along an upper surface of the denticle body.

In accordance with another feature, pouring the dressing material comprises pouring the dressing material to a height over the top surface of the negative mold of at least 0.5 millimeters.

In accordance with another feature, prior to pouring the dressing material, the method further includes providing a fabric on the top surface, wherein pouring the dressing material comprises pouring the dressing material over the fabric, wherein the fabric is thereby contained in the cured wound dressing.

In accordance with another feature, after pouring the dressing material, the method further includes applying vibration to the negative mold for a period of time to drive air out of the denticle cavities, thereby allowing the denticle cavities to fill with the dressing material.

In accordance with embodiments of the invention disclosure, there is provided a wound dressing that includes a dressing member made of a supple non-textile material and having a wound-facing side and an outward facing side opposite the wound-facing side. The wound dressing also includes a plurality of microstructure denticles formed on the wound-facing side. Each denticle of the plurality of denticles includes a base portion that joins the denticle to the wound-facing side of the dressing member. The base portion of each denticle has a front and a rear opposite the front. Each denticle further includes a denticle body that extends upward relative to the base portion, and which extends laterally to the rear and beyond the rear of the base portion. The plurality of denticles are arranged on the dressing member such that the denticle body of each one of the plurality of denticles partially overlaps at least one adjacent denticle.

In accordance with another feature, wherein the plurality of denticles are arranged on the wound-facing side of the dressing member in a series of columns along the wound-facing side of the dressing member, and wherein the columns form staggered rows that alternate from column to column.

In accordance with another feature, for each denticle of the plurality of denticles, the denticle body comprises at least one ridge formed in the denticle body which extends from the front of the base portion to the rear along an upper surface of the denticle body.

In accordance with another feature, the at least one ridge is a plurality of ridges, wherein the plurality of ridges are formed by a plurality of concave valleys formed in the denticle body which extend from the front of the base portion to the rear along the upper surface of the denticle body.

Although the invention is illustrated and described herein as embodied in a wound dressing having a micropatterned surface, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one.

The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

“In the description of the embodiments of the present invention, unless otherwise specified, azimuth or positional relationships indicated by terms such as “up”, “down”, “left”, “right”, “inside”, “outside”, “front”, “back”, “head”, “tail” and so on, are azimuth or positional relationships based on the drawings, which are only to facilitate description of the embodiments of the present invention and simplify the description, but not to indicate or imply that the devices or components must have a specific azimuth, or be constructed or operated in the specific azimuth, which thus cannot be understood as a limitation to the embodiments of the present invention. Furthermore, terms such as “first”, “second”, “third” and so on are only used for descriptive purposes, and cannot be construed as indicating or implying relative importance.

In the description of the embodiments of the present invention, it should be noted that, unless otherwise clearly defined and limited, terms such as “installed”, “coupled”, “connected” should be broadly interpreted, for example, it may be fixedly connected, or may be detachably connected, or integrally connected; it may be mechanically connected, or may be electrically connected; it may be directly connected, or may be indirectly connected via an intermediate medium. As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. Those skilled in the art can understand the specific meanings of the above-mentioned terms in the embodiments of the present invention according to the specific circumstances.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a top view of a denticle configuration for use on a wound dressing, in accordance with some embodiments;

FIG. 2 is a side view of a denticle configuration for use on a wound dressing, in accordance with some embodiments;

FIG. 3 is a perspective view of a denticle configuration for use on a wound dressing, in accordance with some embodiments;

FIG. 4 is a view of a wound-facing surface of a wound dressing including a plurality of denticles, in accordance with some embodiments;

FIG. 5 is a detail view of denticles arranged on a wound dressing and the relative locations of the bases of the respective denticles, in accordance with some embodiments;

FIG. 6 is a side cut-away view of a negative mold for creating a wound dressing having denticles, in accordance with some embodiments;

FIG. 7 is a top perspective view of a negative mold for creating a wound dressing having denticles, in accordance with some embodiments;

FIG. 8 is a perspective view of a sectional cut-away view of a portion of a negative mold for creating a wound dressing having denticles, in accordance with some embodiments;

FIG. 9 is a side view of an alternative configuration of a denticle for use on a wound dressing, in accordance with some embodiments;

FIG. 10 is a top view of an alternative configuration of a denticle for use on a wound dressing, in accordance with some embodiments;

FIG. 11 is a front view of an alternative configuration of a denticle for use on a wound dressing, in accordance with some embodiments;

FIG. 12 is a flow chart diagram of a method of making a wound dressing, in accordance with some embodiments; and

FIG. 13 is a side cut-away view of a portion of a cured wound dressing, in accordance with some embodiments.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The inventive disclosure provides a novel and efficient wound dressing that resists the formation of bacterial growth. Specifically, the wound dressing includes a plurality of denticles that are microstructures arranged in micropatterns on the wound-facing surface of the wound dressing, and which are formed in a way that they create a mechanical interference with each other. In some embodiments, the specific shape and arrangement of the denticles can be modelled after denticles that occur on the skin of some shark species. In some embodiments the denticles can have a more artificial, geometric arrangement. Microstructure patterns have been used in marine environments to reduce biofouling of boat and ship hulls. These microstructures, however, tend to be formed such that they extend perpendicularly away from the surface on which they are formed. While there are varying patterns used, even microstructures formed on other microstructures, there is no mechanical interaction among the microstructure because the surface is rigid.

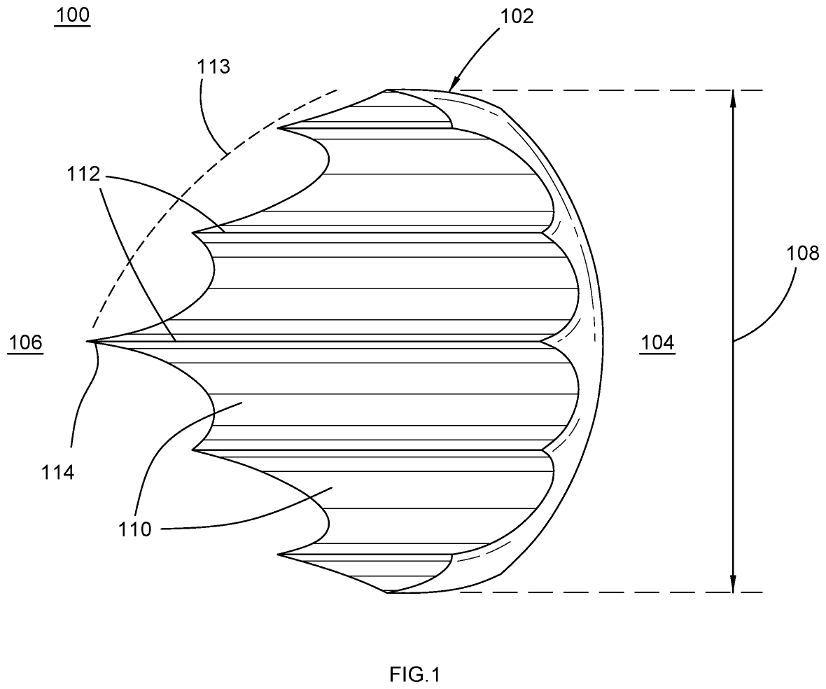

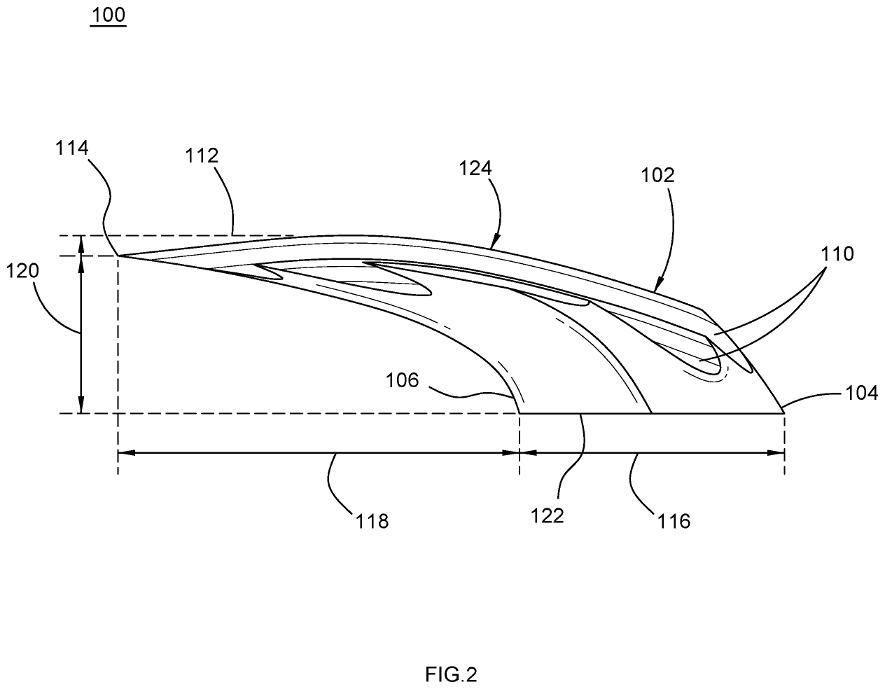

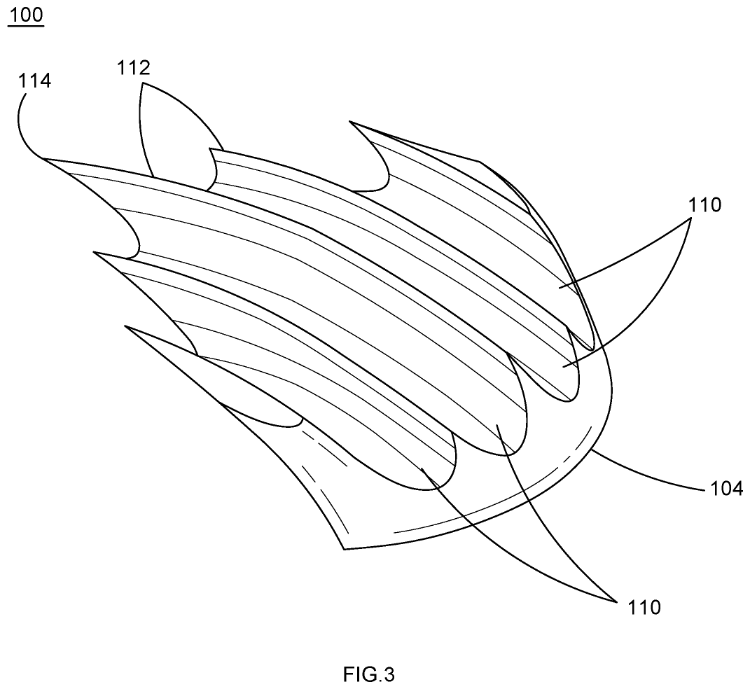

FIG. 1 is a top view of a denticle configuration 100 for use on a wound dressing, in accordance with some embodiments. FIGS. 2 and 3 show, respectively, a side view of the denticle configuration 100, and a perspective view of the denticle configuration 100. The denticle configuration 100 is a configuration used to form a single, microstructure denticle, which is repeated on a wound-facing surface of a wound dressing. As such, the denticle configuration 100 has dimensions on the order of micrometers or micron (μm), to several hundred microns. Further, the denticle configuration 100 is formed to resemble the denticle structure found on the skin of the bonnethead shark, sphyrna tiburo. Those skilled in the art will, however, appreciate that similar denticle structures can be used equivalently, and that the features of denticle structures such as those resembling denticle configuration 100 have an advantage over conventional columnar denticle structures.

The denticle 102 generally has a front 104 and a rear 106. A width 108, from side to side, can be on the order of 50 to 500 microns in some embodiments, but can also be less or more in some embodiments. In FIG. 2, it can be seen that the denticle 102 has a base or base portion 122. The base 122 represents the portion of the denticle that meets with the generally planar surface of the wound dressing, as will be shown. The denticle 102 includes a body 124 that extends from the base 122 to the rear (left side of the page here), and behind a rear 106 of the base 122 by a distance 118. In particular, the body 124 extends upwards from the base 122, and turns to further extend generally laterally to the rear, away from the base 122 and over the surface to which the base is joined. Also, as shown here, the base 122 defines a width 116 from front 104 to rear 106 of the base 122. The body 124 of the denticle 102 can taper, both top to bottom and side to side, to a tip 114, generally. The tip can be located farther (distance 118) to the rear of the base 122 than the width 116 of the base 122. Generally the body 124 extends upward from the base, and arcs to the rear in a substantially horizontal direction. A top surface of the denticle can have a plurality of concave valleys 110 that run from front 104 to the rear, creating ridges 112 between the valleys 110. The ridges 112 form a microstructure on top of the microstructure of the denticle 102. Each of the ridges 112 terminate in a tip like tip 114 at the rear of the denticle body. As seen in the top view of FIG. 1, the front 104 of the denticle can be arcuate in shape, from side to side (e.g. in width 108). Similarly, the rear of the denticle body 124 can have a general arcuate profile, as indicated by broken line 113, and having a central apex at tip 114, with several other tips created on ridges 112 due to the concave valleys 110. In terms of dimensions, the base width 116 from front to rear can be on the order of 20-200 microns, the distance 118 from the rear of the base to the tip 114 can be on the order of 50-500 microns, and the height 120 from the bottom of the base 122 to the tip 114 of the uppermost point of the central ridge can be on the order of 10-225 microns. Dimension values outside of these ranges can also be used.

To form a wound dressing according to the inventive disclosure, a plurality of denticles are formed on at least one side of the wound dressing, and specifically on a wound-facing surface or side. The plurality of denticles are preferably arranged in a pattern where they are close enough to each other and sized/dimensioned to achieve an engineered roughness index of 5-20, which is optimized to inhibit the formation of pathogenic biofilms. That is, the denticles inhibit bioadhesion, and further, by action of lateral mechanical interference with each other, the denticles can potentially dislodge organisms that may have been able to attach to the denticles in spite of the microstructures presenting an inhibitory surface.

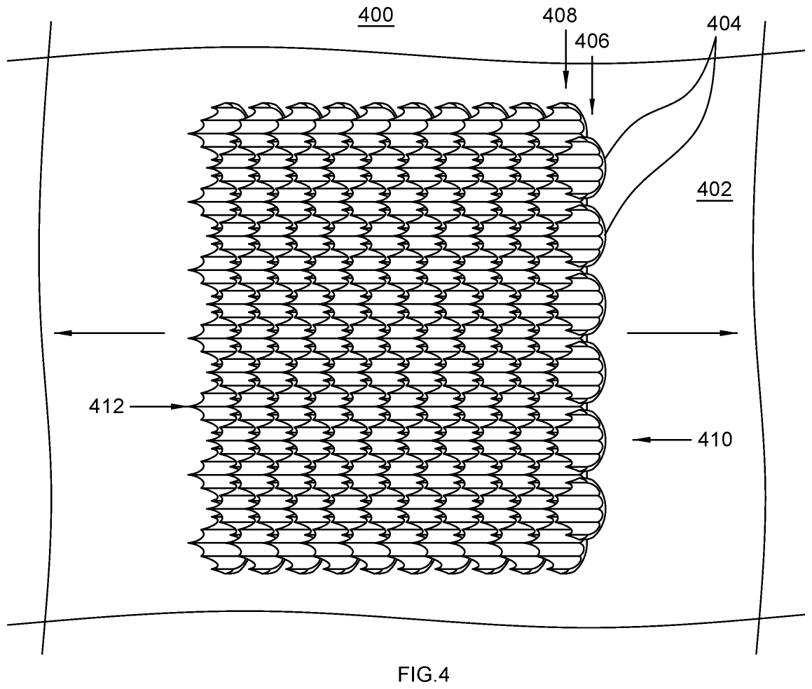

FIG. 4 is a view of a wound-facing surface 402 of a wound dressing 400 including a plurality of denticles 404, in accordance with some embodiments. The denticles 404 can be configured as shown in FIGS. 1-3, or in alternative configurations. Wound dressing 400 and the denticles can be made of the same material or have a material in common. As shown here, the denticles 404 are arranged in columns such as columns 406, 408. The denticles are all commonly oriented, and in each column, the front of each denticle is on one side, and the rear of each denticle is on the opposite side. Thus, within a column, the denticles are arranged end to end with the front to rear axis of each denticle being perpendicular to the direction of the column. The columns are staggered, such that the rows, perpendicular to the direction of the columns, skip consecutive columns. Thus row 410 and row 412, for example, have denticles in alternating columns such that the rows alternate from column to column. So, for example, denticles in row 410 could be in even numbered columns, while denticles in row 412 can be in odd numbered columns. As shown there, the arrangement of denticles can extend to the left and right of those shown (meaning additional columns/rows of denticles which are not shown here), as well as above and below (in the plane of the page as drawn) on the surface of the wound dressing 400. By staggering the rows, the denticles can overlap those in the adjacent column to the rear and be overlapped by those denticles adjacent to the front of a given denticle 404. Thus, when the wound dressing flexes, as can occur due to movement of the patient, the denticles mechanically interfere with those positioned to the rear of a given denticle, further reducing the ability of pathogens to form biofilms through bioadherence by potentially dislodging any organisms that may have attached to the denticles 404.

It will be appreciated by those skilled in the art that alternate arrangements to that shown in FIG. 4 can be used equivalently. For example, although shown here in staggered rows, denticle columns are co-aligned in rows in alternating columns, it is also possible to arrange the denticles in non-staggered, regular rows. However, the degree of overlap among denticles would be diminished. Furthermore, it is contemplated that groups of columns can alternate orientation. That is, several columns can be arranged as shown here, with the rear of each denticle to the left of the page, and other groups of columns can be oriented in the opposite direction such that the rear of the denticles in those columns are arranged to the right of the page. In general, sets of denticles are arranged to create an overlapping arrangement among denticles within the particular set of denticles. Other sets of denticles can be oriented differently. In fact sets of denticles can be arranged in patterns of different orientations. Additionally, it is contemplated that the lateral extending, overlapping denticles shown can be mixed with more conventional columnar type microstructures.

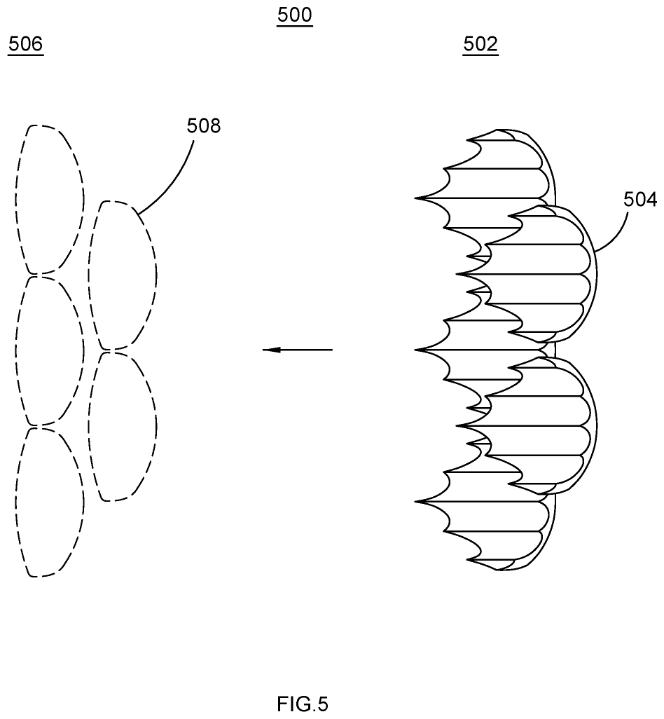

FIG. 5 is a detail view of denticles arranged on a wound dressing and the relative locations of the bases of the respective denticles, in accordance with some embodiments. This figure shows a detail of the arrangement of FIG. 4, with a group 502 of denticles 504 (equivalent to 404) arranged in two columns, with two denticles in the front column and three denticles in the rear column. This pattern can be replicated many times to cover a surface of a wound dressing. As can be seen, the bodies of the denticles in the front column partly extend over the bodies of the denticles in the rear column. Each denticle is independent, however, as indicated by grouping 506, which shows the outlines 508 of the base of each denticle in the group 502. Accordingly, the denticles to the rear of other denticles, in the illustrated arrangement, are partly covered by the denticle bodies of denticles to the front. The bodies of each denticle, however, are free and unattached. As a result, when the wound dressing flexes, such as when the patient moves, the denticles can likewise move relative to each other, providing mechanical interference among the denticles that provides a resistance to the formation of biofilms and bioadherence.

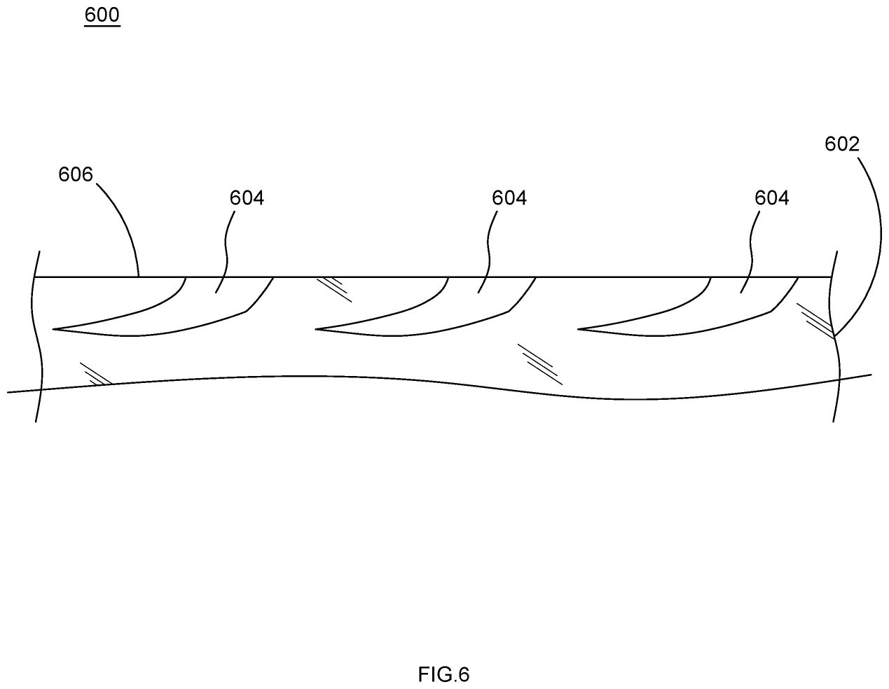

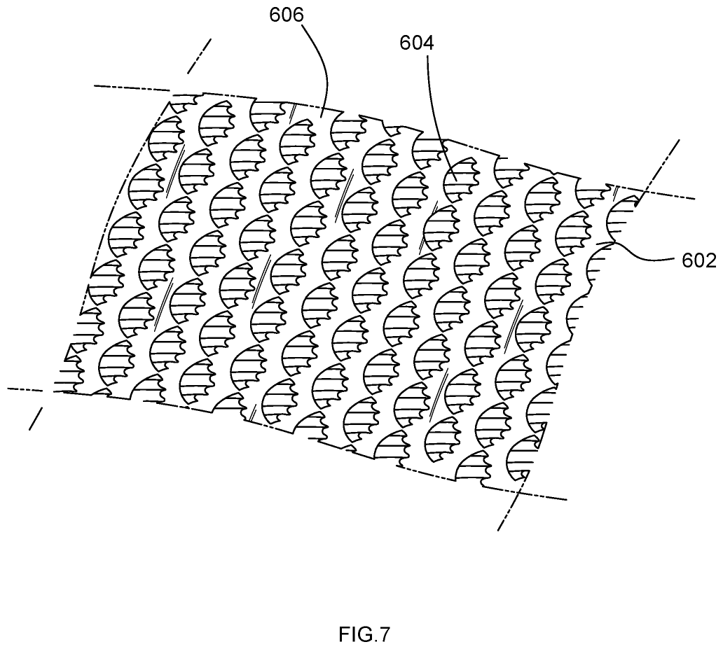

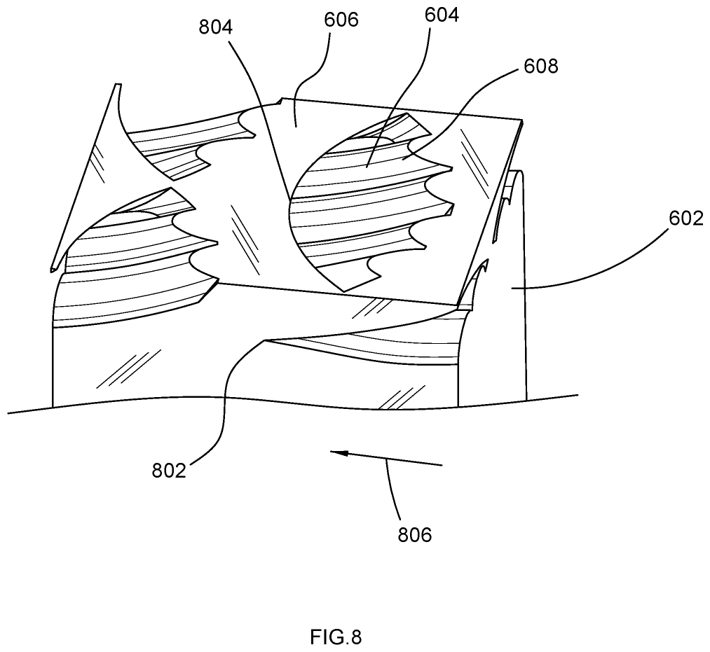

FIGS. 6-8 show various views of a negative mold for forming a wound dressing having a plurality of denticles. FIG. 6 is a side cut-away view of a negative mold for creating a wound dressing having denticles. FIG. 7 is a top perspective view of a negative mold for creating a wound dressing having denticles. FIG. 8 is a perspective view of a sectional cut-away view of a portion of a negative mold for creating a wound dressing having denticles. The negative mold includes a mold body 602 that has a plurality of denticle cavities 604 that open at a top surface 606 and extend into the mold body 602, having the negative shape of a denticle. The mold body can be created by, for example, additive printing techniques. In the particular embodiment shown, in vitro shark skin (S. tiburo) was scanned using a SKYSCAN Micro-CT scanner at 34 kV and 188 vA voltage at 6 micron resolution. Software applications such as CT-Analyzer and CT-Volume were used to render scan data, which was then imported into AutoDesk CAD. After visually evaluating various denticles scanned, one with a high resolution was selected and captured. The selected denticle rendering was imported into OnShape Cloud CAD, and the copied and repeated to produce a pattern as shown, for example, in FIG. 4. A negative mold rendering was then created by positioning the negative (empty) spaces for each denticle cavity 604 corresponding to the selected denticle rendering at the surface 606 of the mold body 602 such that the base of each denticle rendering resulted in a denticle cavity 604 that is open at the top surface 606 of the mold body 602, with the denticle cavity 604 extending into the mold body 602 in the negative shape of the selected denticle. Thus, each denticle cavity is configured to form a corresponding denticle. As can be seen in FIG. 7, the denticle cavities 604 are arranged in columns and staggered rows. In FIG. 8, it can be seen that the denticle cavities 604 of one column overlap those of the adjacent column (e.g. to the rear). The tip 802 of one denticle cavity, for example, overlaps an adjacent denticle cavity, and extends to a position that is about in line with the rear 804 of the overlapped denticle cavity. Also, in FIG. 8, the convex humps 608 can be seen which for the concave valleys (e.g. 112) in each denticle.

Once a negative mold is created, it can be arranged so that the top surface 606 is facing up, and level. The negative mold 600 can be surrounded with release barriers that prevent dressing material from running off the mold 600. The dressing material can include an elastomer, such as, for example, a mixture of polydimethylsiloxane resin and polymethylsiloxane (10:1 mix), which after stirring and degassing can be poured over the negative mold 600 and allowed to cure. Heating may be used to facilitate curing. In some embodiments mechanical vibration may be applied to the mold 600 in order to ensure flow of the dressing material into each of the denticle cavities and to eliminate trapped air by driving the air out of the denticle cavities. The mechanical vibration can be ultrasonic vibration applied for a selected period of time after the dressing material is poured onto the mold 600. Further, prior to pouring the elastomer material, a fabric layer can be placed over the top surface of the mold in order to add strength to the wound dressing that can prevent tearing of the wound dressing.

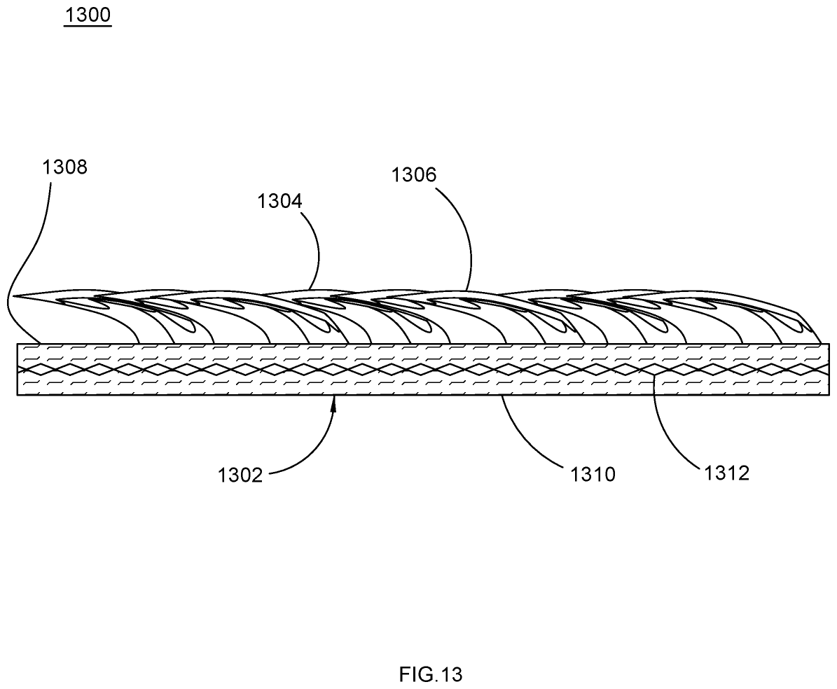

Once the dressing material cures, the cured wound dressing can be peeled or otherwise removed from the mold 600. A portion of a cured wound dressing produced using a mold such as mold 600 is shown in FIG. 13. The cured elastomer produces a dressing member 1302. The elastomer can be a supple non-textile material and having a wound-facing side 1308 and an outward facing side 1310 opposite the wound-facing side 1308. The dressing member 1302 can also include a fabric layer 1312. The denticle cavities 604 of the mold 600 produce a plurality of denticles such as denticles 1304, 1306, which are arranged in columns having staggered row from column to column, and wherein each row has a denticle on every other column (e.g. alternating). Thus denticles 1304, 1306 can be in adjacent rows, such that denticle 1304 appears behind denticle 1306 here. Further, as indicated in FIG. 5, the denticles can overlap other denticles in adjacent columns.

In development of the disclosed wound dressing, the inventor conducted several tests using surfaces having denticles such as that shown in FIGS. 1-5, of varying scale relative to that of the denticles in the skin sample of a bonnethead shark (S. tiburo), from 25% to 125%, in 25% increments. Both migration tests and adhesion tests were conducted. The dimensions for each case are shown in the table of FIG. 15, where “h” is height of the denticle, “d” is the distance between denticles, and “s” is the spacing between denticles. As can be seen, the actual shark skin was calculated to have a Wenzel's Roughness Factor (WRF) of 2.622, and an Engineered Roughness Index (ERI) of 15.313. The 100% artificially created denticles, arranged as shown in FIGS. 4-5, for example, had a WRF of 2.718 and 8.966, respectively. For the migration test these samples were tested for pathogen migration by preparing an agar sample that was divided with the samples bridging the divide. A commercial bandage and a flat elastomeric member were used as controls. At one end of each sample a culture of bacteria was placed. Both Staphylococcus aureus and Staphylococcus epidermis were tested. The test units were incubated at 37 deg. C for 24 hours, and then migration of bacteria across the samples and controls was evaluated to determine the amount of the each sample and control that had been contaminated. The results indicated that the samples created using the arranged denticles experienced substantially less contamination relative to the controls. In particular the 75%, 100%, and 125% scale samples resulted in approximately one quarter to one third of the contamination relative to the controls using a two-way analysis of variance (ANOVA) examining percent. For the adhesion tests the retention of Staphylococci on each surface was tested. It was found that dressings with an engineered roughness index of 5.687 μm, 8.966 μm, and 16.206 μm significantly reduced the adhesion of Staphylococcus.

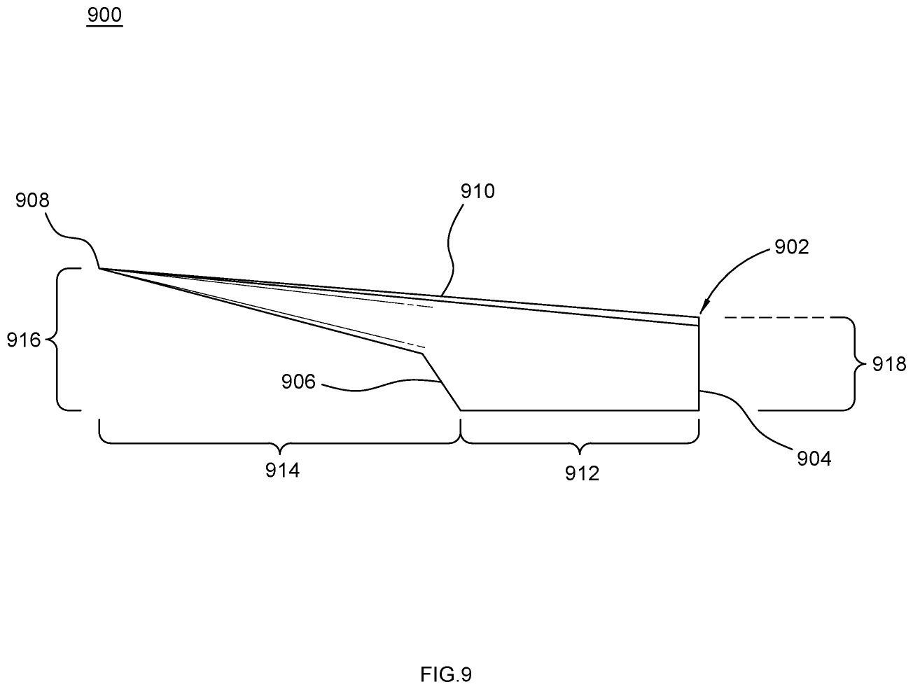

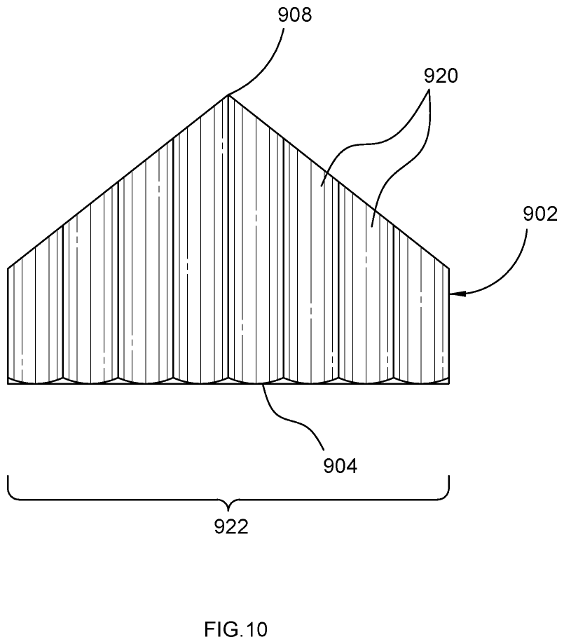

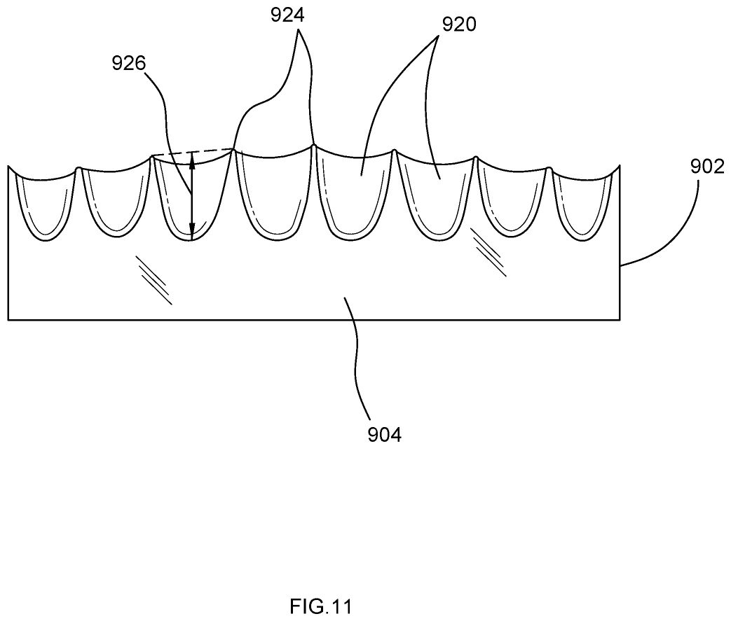

FIGS. 9-11 show an alternative denticle configuration. FIG. 9 is a side view of an alternative configuration of a denticle, FIG. 10 is a top view of the alternative configuration, and FIG. 11 is a front view of the alternative configuration. The alternative denticle configuration is similar to that of FIGS. 1-3, and includes a denticle body including a base portion 902 having a front 904 and a rear 906. The denticle body extends from the base portion 902 to a tip 908 that is to the rear of the base. Further, the portion 910 of the body that extends rearward can also tap to the tip 908 from side to side, and top to bottom. The base can have a width 912 from front 904 to rear 906 that is smaller than the distance 914 from the rear 906 to the tip 908. A height 916 at the tip 908 can be higher than a height 918 at the front 904 to allow overlap among commonly oriented denticles. The top of the denticle body can include several ridges, which can be formed by valleys or troughs such as concave troughs 920 which run from front to rear along the top surface. Other forms of ridges can be formed equivalently. The inter-trough distance, meaning the distance from one trough to the next, or one ridge 924 to the next can be on the order of 2-20 microns. Furthermore, the depth 926 of the troughs, from the top of the adjacent ridges 924 to the bottom of the trough, can be on the order of 2-20 microns. Dimension values outside of these ranges can also be used. The denticle can also have a width 922, from side to side, that is in the range of that for the denticle of FIGS. 1-3. The denticle 900 can be repeated on a wound-facing side of a wound dressing in a pattern that produces a desired engineered roughness index sufficient to resist bioadhesion of pathogens.

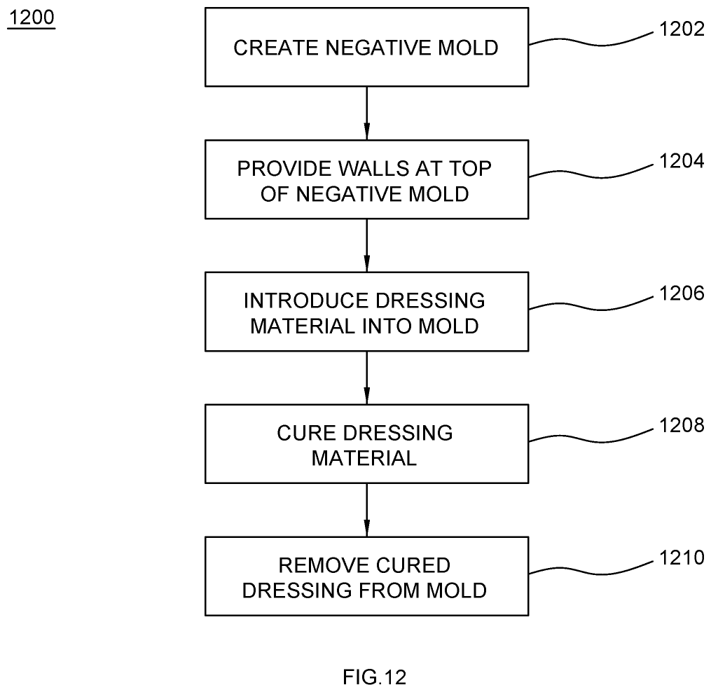

FIG. 12 is a flow chart diagram of a method 1200 of making a wound dressing, in accordance with some embodiments. In step 1202 a negative mold is created that includes a top surface with denticle cavities that are open at the top surface, and which extend into the mold body. The denticle cavities are sized and arranged to achieve a desired bioadherence resistance, as indicated by having an engineered roughness index in a selected range. The denticles produced by the denticle cavities have dimensions on the order of microns to hundreds of microns, and are arranged in a selected pattern that be regular or irregular. The denticle cavities can be configured to produce denticles that are regularly shaped (e.g. square, semi-spherical), or irregularly shaped (e.g. a shark skin denticle). Further, the denticle cavities can be configure to commonly orient irregularly shaped denticles, as well as to arrange them in a repeating pattern.

In step 1204 the negative mold can be placed into a mold tool that provides a barrier around the negative mold to confine dressing material. In step 1206 the dressing material can be introduced into the mold tool. The dressing material can include an elastomeric material, and can further include a strengthening materials such as a fabric or similar material. In step 1208 the dressing material is cured over time, using heat to facilitate curing. In step 1210, the cured wound dressing can be removed from the mold and packaged for subsequent use. The process 1200 creates wound dressings that resist the formation of biofilms, thereby reducing the likelihood of the patient experiencing an infection at the covered wound. The microstructure on the wound-facing side of the wound dressing interferes with the ability of pathogens to adhere to the dressing. In embodiments where the denticles create a mechanical interference with each other, the ability of pathogens to establish a biofilm is even more reduced if the patient moves, causing even a small flex in the wound dressing.

The disclosed wound dressing provides an advantage over both conventional wound dressings and those using conventional columnar microstructures due to the lateral mechanical interference between denticles that results by flex movement of the wound dressing. Further, the denticles, by overlapping, substantially cover the surface of the wound dressing, and in effect, the top portions of the denticles, in the aggregate, become the surface of the wound dressing. The additional micro structures in the form of ridges and troughs on the top of the denticles provide further bioadhesion resistance.

1-7. (canceled)

8. A method of forming a wound dressing, comprising:
forming a negative mold having a top surface with a plurality of denticle cavities formed in the surface and extending in a body of the negative mold, each denticle cavity of the plurality of denticle cavities configured to form a denticle having:
a base portion at an opening in the top surface of the negative mold, and having a front and a rear;
a denticle body that extends from the base portion to the rear beyond the rear of the base portion and is separated from the top surface of the negative mold;
placing a fabric layer on the top surface of the negative mold;
after placing the fabric layer, pouring a dressing material onto the mold that fills in the plurality of denticle cavities to a selected height above the top surface of the negative mold;
curing the dressing material to produce a cured wound dressing, wherein the fabric is thereby contained in the cured wound dressing; and
removing the cured wound dressing from the negative mold, wherein the cured would dressing includes a plurality of denticles corresponding to the plurality of denticle cavities.
prior to pouring the dressing material, providing a fabric on the top surface, wherein pouring the dressing material comprises pouring the dressing material over the fabric, wherein the fabric is thereby contained in the cured wound dressing

9. The method of claim 8, wherein pouring the dressing material comprises pouring an elastomer material.

10. The method of claim 9, wherein pouring the elastomer material comprises pouring polydimethylsiloxane elastomer.

11. The method of claim 8, wherein forming the negative mold comprises forming the plurality of denticle cavities in staggered rows in the top surface of the negative mold.

12. The method of claim 8, wherein forming the negative mold comprises forming the plurality of denticle cavities such that, for each denticle cavity the corresponding denticle formed by the denticle cavity comprises at least one ridge formed in the denticle body which extends from the front of the base portion to the rear along an upper surface of the denticle body.

13. The method of claim 8, wherein forming the negative mold comprises forming the plurality of denticle cavities such that, for each denticle cavity the corresponding denticle formed by the denticle cavity comprises a plurality of ridges, wherein the plurality of ridges are formed by a plurality of concave valleys formed in the denticle body which extend from the front of the base portion to the rear along an upper surface of the denticle body.

14. The method of claim 8, wherein pouring the dressing material comprises pouring the dressing material to a height over the top surface of the negative mold of at least 0.5 millimeters.

15. (canceled)

16. The method of claim 8, further comprising, after pouring the dressing material, applying vibration to the negative mold for a period of time to drive air out of the denticle cavities, thereby allowing the denticle cavities to fill with the dressing material.

17-20. (canceled)

21. A method of forming a wound dressing, comprising:
forming a negative mold having a top surface with a plurality of denticle cavities formed in the surface and extending in a body of the negative mold, each denticle cavity of the plurality of denticle cavities configured to form a denticle having:
a base portion at an opening in the top surface of the negative mold, and having a front and a rear;
a denticle body that extends from the base portion to the rear beyond the rear of the base portion and is separated from the top surface of the negative mold;
pouring a dressing material onto the mold that fills in the plurality of denticle cavities to a selected height above the top surface of the negative mold;
while pouring the dressing material, applying vibration to the negative mold and continuing to apply the vibration for a period of time after pouring the dressing material to drive air out of the denticle cavities, thereby allowing the denticle cavities to fill with the dressing material;
curing the dressing material to produce a cured wound dressing; and
removing the cured wound dressing from the negative mold, wherein the cured would dressing includes a plurality of denticles corresponding to the plurality of denticle cavities.

22. The method of claim 21, wherein pouring the dressing material comprises pouring an elastomer material.

23. The method of claim 22, wherein pouring the elastomer material comprises pouring polydimethylsiloxane elastomer.

24. The method of claim 21, wherein forming the negative mold comprises forming the plurality of denticle cavities in staggered rows in the top surface of the negative mold.

25. The method of claim 21, wherein forming the negative mold comprises forming the plurality of denticle cavities such that, for each denticle cavity the corresponding denticle formed by the denticle cavity comprises at least one ridge formed in the denticle body which extends from the front of the base portion to the rear along an upper surface of the denticle body.

26. The method of claim 21, wherein forming the negative mold comprises forming the plurality of denticle cavities such that, for each denticle cavity the corresponding denticle formed by the denticle cavity comprises a plurality of ridges, wherein the plurality of ridges are formed by a plurality of concave valleys formed in the denticle body which extend from the front of the base portion to the rear along an upper surface of the denticle body.

27. The method of claim 21, wherein pouring the dressing material comprises pouring the dressing material to a height over the top surface of the negative mold of at least 0.5 millimeters.
